(19) United States
(12) Patent Application Publication
(10) Pub. No.: US 2023/0124856 A1
(43) Pub. Date: Apr. 20, 2023

(54) GENOME EDITING IN SUNFLOWER

(71) Applicant: PIONEER HI-BRED INTERNATIONAL, INC., JOHNSTON, IA (US)

(72) Inventors: GEORGE J. HOERSTER, DES MOINES, IA (US); SANDEEP KUMAR, JOHNSTON, IA (US); BRIAN L LENDERTS, JOHNSTON, IA (US); KEITH S LOWE, JOHNSTON, IA (US)

(73) Assignee: PIONEER HI-BRED INTERNATIONAL, INC., JOHNSTON, IA (US)

(21) Appl. No.: 17/907,040

(22) PCT Filed: Mar. 23, 2021
(86) PCT No.: PCT/US21/23643

§ 371 Date: Sep. 22, 2022

Related U.S. Application Data
(60) Provisional application No. 63/000,528, filed on Mar. 27, 2020.

(51) Int. Cl. C12N 15/82; C12N 15/11; C12N 9/22

(52) U.S. Cl. CPC C12N 15/8247; C12N 15/8205; C12N 15/8213; C12N 15/11; C12N 9/22; C12N 2310/20; C12N 2800/80

(57) ABSTRACT

Compositions and methods for genome editing in sunflower are provided. Exemplary compositions include constructs for Cas endonuclease mediated genome editing of the FAD2-1 locus as well as sunflower plants, seeds and cells thereof which are modified using the disclosed compositions.

### CROSS REFERENCE TO RELATED APPLICATIONS

This application claims the benefit of priority to U.S. Provisional Patent Application No. 63/000,528, filed Mar. 27, 2020, the entire content of which is herein incorporated by reference.

### REFERENCE TO SEQUENCE LISTING SUBMITTED ELECTRONICALLY

The official copy of the sequence listing is submitted electronically via EFS-Web as an ASCII formatted sequence listing with a file named 8204-US-PSP_ST25 created on 25 Mar. 2020 and having a size of 7 kilobytes and is filed concurrently with the specification. The sequence listing comprised in this ASCII formatted document is part of the specification and is herein incorporated by reference in its entirety.

### BACKGROUND

To meet the challenge of increasing global demand for food production, many effective approaches to improving agricultural value (e.g. improved nutritional quality, increased yield, pest or disease resistance, drought and stress tolerance, herbicide resistance, production of industrially useful compounds and/or materials from the plant, and/or production of pharmaceuticals) rely on either mutation breeding or introduction of novel genes into the genomes of crop species by transformation. Both processes are inherently non-specific and relatively inefficient. For example, conventional plant transformation methods deliver exogenous DNA that integrates into the genome at random locations. The random nature of these methods makes it necessary to generate and screen hundreds of unique random-integration events per construct in order to identify and isolate transgenic lines with desirable attributes. Moreover, conventional transformation methods create several challenges for transgene evaluation including: (a) difficulty for predicting whether pleiotropic effects due to unintended genome disruption have occurred; and (b) difficulty for comparing the impact of different regulatory elements and transgene designs within a single transgene candidate, because such comparisons are complicated by random integration into the genome. As a result, conventional plant trait engineering is a laborious and cost intensive process with a low probability of success.

Precision gene modification overcomes the logistical challenges of conventional practices in plant systems. Methods and compositions for targeted cleavage of genomic DNA can be used, for example, to induce targeted mutagenesis, induce targeted deletions of cellular DNA sequences, and facilitate targeted recombination and integration at a predetermined chromosomal locus. See, for example, Umov et al. (2010) Nature 435(7042): 646-51; United States Patent Publications 20030232410; 20050208489; 20050026157; 20050064474; 20060188987; 20090263900; 20090117617; 20100047805; 20110207221; 20110301073; 2011089775; 20110239315; 20110145940; and International Publication WO 2007/014275. Cleavage can occur through the use of specific nucleases such as engineered zinc finger nucleases (ZFN), transcription-activator like effector nucleases (TALENs), or using the CRISPR-Cas system with an engineered crRNA/tracr RNA (‘single guide RNA’) to guide specific cleavage.

Sunflower is a global oil crop that has high potential for climate change adaptation. Cultivated sunflower maintains stable yields across a wide variety of environmental conditions including drought. The narrow margin between demand and supply of oil crops suggests a healthy market situation for oil crops. In addition, demand for high-oleic vegetable oils is rising due to consumer demand for food ingredients that are associated with improved nutritional quality and increasing industrial applications such as environmentally sustainable biodiesel (Wilson (2012) J. Oleo Sci 61(7): 357-67). Mining of resistance alleles from compatible wild sunflower relatives provides further potential for improved agronomics and climate resilience (Badouin et al. (2017) Nature 546: 148-52). Genome editing can be a key tool for bringing these quality agronomic traits in expedited timelines (Chen et al., (2019) Annu. Rev. Plant Biol. 70: 667-97). Accordingly, there is a desire for an effective gene editing system, such as CRISPR-Cas-mediated genome editing, that provides a fast-track platform to make these improvements in sunflower. The content and disclosure of each publication, patent document, and other reference cited in this application is incorporated herein by reference in its entirety for all purposes.

### BRIEF SUMMARY OF INVENTION

Provided herein are compositions and methods for genome editing in sunflower. The disclosure provides compositions and methods for altering the fatty acid profile in sunflower by introducing at least one nucleic acid modification via targeted DNA breaks at a genomic locus of a plant. In one example, the targeted genomic modification is directed to FAD2-1 locus polynucleotide SEQ ID NO:1. In some cases, the oleic acid content in the seed is increased compared to the seed of a control plant not comprising the one or more introduced nucleic acid modifications. Modifications can be introduced using targeted genome editing techniques to create targeted DNA breaks in the genome.

In one aspect, the disclosure provides a method of using genome editing to alter a target site in the genome of a sunflower plant. The method includes the use of a site-specific endonuclease to create DNA breaks that lead to modifications at the sunflower genome target site. The method can include providing a recombinant DNA construct encoding a site-specific endonuclease that, when expressed, causes a targeted DNA break in the plant cell's genome.

In one aspect, the disclosure provides a method of modifying a target site in the genome of a sunflower plant cell, which method includes providing a recombinant DNA construct comprising a nucleic acid sequence encoding one or more guide RNAs to a sunflower plant cell having a Cas endonuclease; and expressing the one or more guide RNAs to form a complex with Cas endonuclease that enables the Cas endonuclease to introduce a double strand break at a target site in the plant cell's genome. The targeted DNA break can be repaired via an imperfect repair that introduces a targeted modification at the target site. The targeted modification can be an insertion, deletion, or substitution of one or more nucleotides at the sunflower genome target site. For example, the Cas endonuclease-induced double stranded break can be used to introduce a modification at the target site that results in one or more of the following: reduced expression of a polynucleotide encoding a polypeptide; reduced activity of a polypeptide; generation of one or more alternative spliced transcripts of a polynucleotide encoding a polypeptide; deletion of one or more active sites of a polypeptide; frameshift mutation in one or more exons of a polynucleotide encoding a polypeptide; deletion of a substantial portion or the full length of a polynucleotide encoding a polypeptide; repression of an enhancer motif present within a regulatory region operably linked to a coding sequence for a polypeptide; or modification of one or more nucleotides of a regulatory element in a promoter, intron, 3′UTR, or terminator that is operably linked to the expression of the polynucleotide encoding the polypeptide.

In one example of the foregoing method of modifying a target site the genome of a sunflower plant cell, the modified target site is in a fatty acid desaturase (FAD) locus involved in sunflower fatty acid metabolism. Exemplary sunflower FAD loci include FAD2 and FAD3 genes. The nucleic acid modifications may be targeted to a FAD 2 or FAD 3 coding region; non-coding region; regulatory sequence; or untranslated region. In some examples, the target sequence is the FAD2-1 gene and the double strand break is induced by a Cas endonuclease and two guide RNAs having sequences SEQ ID NO:2 and SEQ ID NO:3. The modified genomic locus can be a truncated FAD2-1 polypeptide, a non-translatable transcript of the polynucleotide encoding FAD2-1, a non-functional FAD2-1 polypeptide, a pre-mature stop codon, or any combination thereof that result in (i) reduced (or absence of) expression of a polynucleotide encoding the FAD2-1 polypeptide or (ii) reduced (or absence of) activity of the FAD2-1 polypeptide.

The foregoing method can further include generating a sunflower plant having the modified target site in its genome. In one example, the method includes targeted modification of the endogenous FAD2-1 gene sequence SEQ ID NO:1 or a sequence that is at least 90%, at least 91%, at least 92%, at least 93%, at least 94%, at least 95%, at least 96%, at least 97%, at least 98%, or at least 99% identical to SEQ ID NO:1. A plant having modified FAD2-1 locus can produce seed having increased oleic acid content relative to seed from a control sunflower plant. Additionally, the targeted modification can be stably incorporated into the plant's genome, such that the modification can be stably transmitted from parent to progeny plants. In preferred aspects of a sunflower plant, the targeted modification and modified fatty acid profile does not substantially affect the seed yield of the gene edited sunflower plant as compared to a control sunflower plant. In accordance with the foregoing, the disclosure provides plants and seeds in which the FAD gene locus has been modified pursuant to the methods disclosed herein.

In a different example of the foregoing method of modifying a target site the genome of a sunflower plant cell using Cas9 and guide RNAs, the target site is modified by delivery of a heterologous nucleic acid sequence of interest which is integrated into the genome at the site of interest. The integrated nucleic acid sequence of interest can be a fatty acid metabolism gene, carbohydrate metabolism gene, insecticidal resistance gene, herbicidal tolerance gene, nutritional quality gene, yield gene, pest or disease resistance gene, drought tolerance gene, stress tolerance gene, nitrogen use efficiency gene, or water use efficiency gene. The nucleic acid sequence of interest can be integrated into a FAD2 gene, e.g., a FAD2-1 gene sequence SEQ ID NO:1 or a sequence that is at least 90%, at least 91%, at least 92%, at least 93%, at least 94%, at least 95%, at least 96%, at least 97%, at least 98%, or at least 99% identical to SEQ ID NO:1; and the method can include the use of two guide RNAs comprising sequences SEQ ID NO:2 and SEQ ID NO:3, respectively. This method can further include generating a sunflower plant and seed thereof having the nucleic acid sequence of interest can be integrated into the target site.

The disclosure also provides a method of detecting the presence of a polynucleotide comprising modified SEQ ID NO:1 and which is indicative of the presence of a deletion or other modification in a FAD2-1 gene. In one example, the method includes providing a sample of genomic sunflower DNA, contacting the sample with (i) first and second DNA primers comprising SEQ ID NOs:6 and 7 respectively and (ii) first and second DNA probes comprising SEQ ID NO:8 and 12 respectively, and performing an amplification reaction to produce an amplicon. The first probe can be used to detect the amplicon, wherein detecting the amplicon indicates the presence of a wild-type sequence at the CR3 target site of the FAD2-1 gene, while a decreased detection signal or absence of a detection signal indicates a disrupted or modified CR3 target site in the FAD2-1 gene. A decreased detection signal by the first probe (SEQ ID NO:8) at the disrupted target site is determined when compared to the detection signal of a second probe (SEQ ID NO:12) which is designed downstream of the CR3 target site. In another example, genomic sunflower DNA sample is contacting with (i) first and second DNA primers comprising SEQ ID NOs:9 and 10, respectively, and (ii) first and second DNA probes comprising SEQ ID NO:11 and 13 respectively. The first probe can be used to detect the amplicon, wherein detecting the amplicon indicates the presence of a wild-type sequence in the CR4 target site of the FAD2-1 gene, while a decreased detection signal or absence of a detection signal indicates a disrupted or modified CR4 target site in the FAD2-1 gene. A decreased detection signal by the wild-type probe (SEQ ID NO:11) at the disrupted target site is determined when compared to the detection signal of a CR4 standard probe (SEQ ID NO:13) which is designed upstream of the CR4 target site.

The disclosure provides a method of detecting the presence of a modification in a FAD2-1 allele (SEQ ID NO:1). The method includes providing a plurality of samples comprising sunflower genomic DNA, contacting the sample with a pair of DNA primers and two probes, and then performing a nucleic acid amplification reaction to generate an amplicon. In a first example, the pair of primers includes a first and second primer comprising SEQ ID NO:6 and SEQ ID NO:7, respectively, such that detection of an amplicon using a wild-type probe comprising SEQ ID NO: 8 indicates the presence of a wild-type sequence in the CR3 target site of the FAD2-1 gene, while a decreased detection signal or absence of a detection signal indicates a disrupted or modified CR3 target site in the FAD2-1 gene. A decreased detection signal by the wild-type probe (SEQ ID NO: 8) at the disrupted target site is determined when compared to the detection signal of a CR3 standard probe (SEQ ID NO:12) which is designed downstream of the CR3 target site. In a second example, the first and second primers comprise SEQ ID NO:9 and SEQ ID NO:10, respectively, such that detection of an amplicon using a wild-type probe comprising SEQ ID NO:11 indicates the presence of a wild-type sequence in the CR4 target site of the FAD2-1 gene, while a decreased detection signal or absence of a detection signal indicates a disrupted or modified CR4 target site in the FAD2-1 gene. A decreased detection signal by the wild-type probe (SEQ ID NO:11) at the disrupted target site is determined when compared to the detection signal of a CR4 standard probe (SEQ ID NO:13) which is designed upstream of the CR4 target site.

The foregoing and other features will become more apparent from the following detailed description of several embodiments, which proceeds with reference to the accompanying figures.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 depicts a map of sunflower FAD2-1 gene sequence used for genome editing.

FIG. 2 depicts a vector map of sunflower transformation construct used for genome editing.

FIG. 3 depicts illustrative sequence modifications for Plant 1 at the CR3 target site.

FIG. 4 depicts illustrative sequence modifications for Plant 2 at the CR4 target site.

FIG. 5 depicts illustrative sequence modifications for Plant 2 at the CR3 target site.

SEQ ID NO:1 is a nucleic acid sequence of Helianthus annuus FAD2-1 gene.

SEQ ID NO:2 is the nucleic acid sequence of a CR3 guide RNA.

SEQ ID NO:3 is the nucleic acid sequence of a CR4 guide RNA.

SEQ ID NO:4 is the nucleic acid sequence for Arabidopsis thaliana U6 promoter (AT-U6-PRO).

SEQ ID NO:5 is the nucleic acid sequence for Arabidopsis thaliana UBIQ10 promoter (AT-UBIQ10-PRO).

SEQ ID NO:6 is a forward primer for a qPCR assay to detect gene edited modifications at a CR3 target site. It is also a NGS primer to analyze mutated sequences at this target site.

SEQ ID NO:7 is a reverse primer for a qPCR assay to detect gene edited modifications at a CR3 target site. It is also a NGS primer to analyze mutated sequences at the target site.

SEQ ID NO:8 is a probe for a qPCR assay to detect wild-type sequence at a CR3 target site.

SEQ ID NO:9 is a forward primer for a qPCR assay to detect gene edited modifications at a CR4 target site. It is also a NGS primer to analyze mutated sequences at this target site.

SEQ ID NO:10 is a reverse primer for a qPCR assay to detect gene edited modifications at a CR4 target site. It is also a NGS primer to analyze mutated sequences at the this site.

SEQ ID NO:11 is a probe for a qPCR assay to detect wild-type sequence at a CR4 target site.

SEQ ID NO:12 is a CR3 standard probe for a qPCR assay to detect wild-type sequence at a CR3 target site.

SEQ ID NO:13 is a CR4 standard probe for a qPCR assay to detect wild-type sequence at a CR4 target site.

### DETAILED DESCRIPTION

The disclosure provides various embodiments of an approach for targeted modifications of nucleic acids in a host sunflower genome. The disclosed methods can be used to create targeted genome edited modifications in a sunflower plant cell or seed thereof that do not significantly impact agronomic phenotypes of the modified sunflower, other than phenotypes controlled by the genome edited modification.

### I. FAD2

Disclosed herein is a method of targeted genomic modification of loci designated as fatty acid desaturases (FADs) in sunflower. FAD loci are quantitative trait loci (QTL) involved in the inheritance of the complex multigenic trait of fatty acid content in plants. Within the plant oil biosynthetic pathway, FAD genes play a key role in plant lipid biosynthesis and their activity significantly influences the fatty acid composition. FADs are abundant in plants, and expression analysis suggested that FAD mRNAs are produced in over-abundance. Furthermore, FAD genes are expressed in various, tissues, and cell types, as well as subcellular compartments including the plastid and endoplasmic reticulum.

The fatty acid composition of plants, and the performance of oils produced therefrom in many applications, is determined by the relative concentrations of the major fatty acid constituents; oleic (C18:1), linoleic (C18:2), and linolenic (C18:3). The concentrations of these fatty acids are predominantly regulated by the function of the enzymes FAD2 and FAD3. FAD2 encodes the enzyme responsible for the desaturation of oleic acid to linoleic acid. Tanhuanpaa et al. (1998) Mol. Breed. 4: 543-50; Schierholt et al. (2001) Crop Sci. 1: 1444-9. Oleic acid is converted to linoleic acid and linolenic acid in plants according to the scheme:

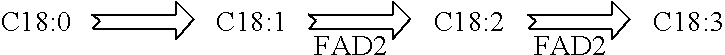

FAD2 genes have been identified in major plant and algal species including but not limited to maize, soybean, cotton, Arabidopsis, wheat, forage grasses, rice, sunflower and Brassica, and modification of FAD2 expression leads to altered fatty acid profiles in such organisms. Furthermore, plants comprising modified FAD2 genes have been commercialized, and disruption of a FAD2 gene has been shown to be able to improve the nutritional and functional properties of oil produced by a host plant without an agronomic penalty to the host plant. For example, canola and sunflower varieties that have been commercialized under the Nexera® brand (Dow AgroSciences, LLC) are characterized by a higher oleic acid, lower linoleic acid, and lower linolenic acid (and lower saturated fatty acid) composition, when compared to wild-type canola and sunflower profiles. Nexera® brand canola and sunflower varieties have been developed through conventional plant breeding techniques.

FAD2 loci may be modified and/or disrupted in a plant without detrimentally affecting the value of the plant, and for many purposes, with an actual increase in its value, including alteration of FAD2 expression, alteration of oil content/ratios and or integration and expression of desired transgenes. Furthermore, according to the ubiquitous nature of FAD loci in plants, FAD2 loci may be modified and or disrupted without detriment for at least some purposes in many species, including, for example and without limitation: canola; soybean; maize; wheat; forage grasses; Brassica sp.; rice, tomatoes, barley; oats; sorghum; cotton; and sunflower, as well as fungi and algae. In some embodiments disclosed herein, the endogenous sunflower FAD2-1 locus (SEQ ID NO:1) is modified. In some examples, the FAD2-1 locus is used as a target site for site-specific DSBs, which result in a modified locus. For example, the site-specific DSBs may modify the locus so as to produce a disrupted (i.e., inactivated) FAD2-1 gene.

### II. Terms

As used in this application, including the claims, terms in the singular and tie singular forms, “a,” “an,” and “the,” for example, include plural referents, unless the content clearly dictates otherwise. Thus, for example, a reference to “plant,” “the plant,” or “a plant” also refers to a plurality of plants. Furthermore, depending on the context, use of the term, “plant,” may also refer to genetically-similar or identical progeny of that plant. Similarly, the term, “nucleic acid,” may refer to many copies of a nucleic acid molecule. Likewise, the term, “probe,” may refer to many similar or identical probe molecules.

Numeric ranges are inclusive of the numbers defining the range, and expressly include each integer and non-integer fraction within the defined range. Unless defined otherwise, all technical and scientific terms used herein have the same meaning as commonly understood by one of ordinary skill in the art.

In order to facilitate review of the various embodiments described in this disclosure, the following explanation of specific terms is provided.

As used herein, “sunflower” means a plant that produces oil-type sunflower seeds or a plant that produces non-oil type sunflower seeds. Oil-type sunflower seeds include seeds used to produce any sunflower oil type, such as, linoleic, high oleic, or mid oleic oil type. Non-oil type sunflower seeds include food grade or confectioners seed and birdseed. Sunflower includes commercial crop species Helianthus annuus.

As used herein, “locus” is a chromosomal locus or region where a polymorphic nucleic acid, trait determinant, gene, or marker is located. A “locus” can be shared by two homologous chromosomes to refer to their corresponding locus or region. As used herein, “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant allele for a gene may have a reduced or eliminated activity or expression level for the gene relative to the wild-type allele.

As used herein, the term “homozygous” refers to a genotype comprising two identical alleles at a given locus in a diploid genome, or a genotype comprising two non-identical mutant alleles at a given locus in a diploid genome. The latter genotype comprising two non-identical mutant alleles is also referred to as being heteroallelic or transheterozygous, or as a heteroallelic combination. As used herein, “heterozygous” describes a genotype comprising a mutant allele and a wild-type allele at a given locus in a diploid genome.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. For purposes of calculating “percent identity” between DNA and RNA sequences, a uracil (U) of a RNA sequence is considered identical to a thymine (T) of a DNA sequence. If the window of comparison is defined as a region of alignment between two or more sequences (i.e., excluding nucleotides at the 5′ and 3′ ends of aligned polynucleotide sequences, or amino acids at the N-terminus and C-terminus of aligned protein sequences, that are not identical between the compared sequences), then the “percent identity” may also be referred to as a “percent alignment identity”. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

It is recognized that residue positions of proteins that are not identical often differ by conservative amino acid substitutions, where amino acid residues are substituted for other amino acid residues with similar size and chemical properties (e.g., charge, hydrophobicity, polarity, etc.), and therefore may not change the functional properties of the molecule. When sequences differ in conservative substitutions, the percent sequence similarity may be adjusted upwards to correct for the conservative nature of the non-identical substitution(s). Sequences that differ by such conservative substitutions are said to have “sequence similarity” or “similarity.” Thus, “percent similarity” or “percent similar” as used herein in reference to two or more protein sequences is calculated by (i) comparing two optimally aligned protein sequences over a window of comparison, (ii) determining the number of positions at which the same or similar amino acid residue occurs in both sequences to yield the number of matched positions, (iii) dividing the number of matched positions by the total number of positions in the window of comparison (or the total length of the reference or query protein if a window of comparison is not specified), and then (iv) multiplying this quotient by 100% to yield the percent similarity. Conservative amino acid substitutions for proteins are known in the art.

For optimal alignment of sequences to calculate their percent identity or similarity, various pair-wise or multiple sequence alignment algorithms and programs are known in the art, such as ClustalW, or Basic Local Alignment Search Tool® (BLAST®), etc., that may be used to compare the sequence identity or similarity between two or more nucleotide or protein sequences. Although other alignment and comparison methods are known in the art, the alignment between two sequences (including the percent identity ranges described above) may be as determined by the ClustalW or BLAST® algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); and Larkin M A et al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990).

The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

The term “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature—e.g., the promoter or regulatory sequence has a different origin relative to the associated polynucleotide sequence and/or the promoter or regulatory sequence is not naturally occurring in a plant species to be transformed with the promoter or regulatory sequence.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., may comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., may also refer to a polynucleotide or protein molecule or sequence that has been genetically engineered and/or constructed outside of a cell. For example, a recombinant DNA molecule may comprise any engineered or man-made plasmid, vector, etc., and may include a linear or circular DNA molecule. Such plasmids, vectors, etc., may contain various maintenance elements including a prokaryotic origin of replication and selectable marker, as well as one or more transgenes or expression cassettes perhaps in addition to a plant selectable marker gene, etc.

As used herein, the term “isolated” refers to at least partially separating a molecule from other molecules typically associated with it in its natural state. In one embodiment, the term “isolated” refers to a DNA molecule that is separated from the nucleic acids that normally flank the DNA molecule in its natural state. For example, a DNA molecule encoding a protein that is naturally present in a bacterium would be an isolated DNA molecule if it was not within the DNA of the bacterium from which the DNA molecule encoding the protein is naturally found. Thus, a DNA molecule fused to or operably linked to one or more other DNA molecule(s) with which it would not be associated in nature, for example as the result of recombinant DNA or plant transformation techniques, is considered isolated herein. Such molecules are considered isolated even when integrated into the chromosome of a host cell or present in a nucleic acid solution with other DNA molecules.

As used herein, an “encoding region” or “coding region” refers to a portion of a polynucleotide that encodes a functional unit or molecule (e.g., without being limiting, a mRNA, protein, or non-coding RNA sequence or molecule).

As used herein, “modified” in the context of a plant, plant seed, plant part, plant cell, and/or plant genome, refers to a plant, plant seed, plant part, plant cell, and/or plant genome comprising an engineered change in the expression level and/or coding sequence of one or more target genes (for example one or more FAD genes), relative to a wild-type or control plant, plant seed, plant part, plant cell, and/or plant genome, such as via (A) a transgenic event comprising a suppression construct or transcribable DNA sequence encoding a non-coding RNA that suppresses a target gene, e.g., a FAD2 gene, for suppression, or (B) a genome editing event or mutation affecting (e.g., reducing or eliminating) the expression level or activity of an endogenous target gene (e.g., a FAD2 gene). Indeed, the term “modified” may further refer to a plant, plant seed, plant part, plant cell, and/or plant genome having one or more mutations affecting expression of one or more endogenous target genes (e.g. a FAD gene, such as an endogenous FAD2 gene) introduced through chemical mutagenesis, transposon insertion or excision, or any other known mutagenesis technique, or introduced through genome editing. For clarity, therefore, a modified plant, plant seed, plant part, plant cell, and/or plant genome includes a mutated, edited and/or transgenic plant, plant seed, plant part, plant cell, and/or plant genome having a modified expression level, expression pattern, and/or coding sequence of one or more target genes (such as one or more FAD genes) relative to a wild-type or control plant, plant seed, plant part, plant cell, and/or plant genome. Modified plants can be homozygous or heterozygous for any given mutation or edit, and/or may be bi-allelic or heteroallelic at the target gene locus. For example, a modified plant is bi-allelic or heteroallelic for a targeted FAD gene if each copy of the FAD gene is a different allele (i.e., comprises different mutation(s) and/or edit(s)), wherein each allele lowers the expression level and/or activity of the FAD gene. Modified plants or seeds may contain various molecular changes that affect expression of target genes (e.g., FAD gene(s), such as a FAD2 gene), including genetic and/or epigenetic modifications. Modified plants, plant parts, seeds, etc., may have been subjected to mutagenesis, genome editing or site-directed integration (e.g., without being limiting, via methods using site-specific nucleases), genetic transformation (e.g., without being limiting, via methods of Agrobacterium transformation or microprojectile bombardment), or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to one or more target genes. A modified seed provided herein may give rise to a modified plant provided herein. A modified plant, plant seed, plant part, plant cell, or plant genome provided herein may comprise a recombinant DNA construct or vector or genome edit as provided herein. A “modified plant product” may be any product made from a modified plant, plant part, plant cell, or plant chromosome provided herein, or any portion or component thereof.

A modified plant, plant part, cell, or explant provided herein may be of an elite variety or an elite line. An elite variety or an elite line refers to a variety that has resulted from breeding and selection for superior agronomic performance. An edited plant, cell, or explant provided herein may be a hybrid plant, cell, or explant. As used herein, a “hybrid” is created by crossing two plants from different varieties, lines, inbreds, or species, such that the progeny comprises genetic material from each parent. Skilled artisans recognize that higher order hybrids can be generated as well. For example, a first hybrid can be made by crossing Variety A with Variety B to create an A×B hybrid, and a second hybrid can be made by crossing Variety C with Variety D to create an C×D hybrid. The first and second hybrids can be further crossed to create the higher order hybrid (A×B)×(C×D) comprising genetic information from all four parent varieties.

As used herein, the term “control plant” (or likewise a “control” plant seed, plant part, plant cell and/or plant genome) refers to a plant (or plant seed, plant part, plant cell and/or plant genome) that is used for comparison to a modified plant (or modified plant seed, plant part, plant cell and/or plant genome) and has the same or similar genetic background (e.g., same parental lines, hybrid cross, inbred line, testers, etc.) as the modified plant (or plant seed, plant part, plant cell and/or plant genome), except for the transgenic and/or genome editing event(s) affecting one or more target genes in the modified plant. For example, a control plant may be an inbred line that is the same as the inbred line used to make the modified plant, or a control plant may be the product of the same hybrid cross of inbred parental lines as the modified plant, except for the absence in the control plant of any transgenic or genome editing event(s) affecting one or more targeted FAD genes. Similarly, an unmodified control plant refers to a plant that shares a substantially similar or essentially identical genetic background as a modified plant, but without the one or more engineered changes to the genome (e.g., transgene, mutation or edit) of the modified plant. For purposes of comparison to a modified plant, plant seed, plant part, plant cell and/or plant genome, a “wild-type plant” (or likewise a “wild-type” plant seed, plant part, plant cell and/or plant genome) refers to a non-transgenic and non-genome edited control plant, plant seed, plant part, plant cell and/or plant genome. As used herein, a “control” plant, plant seed, plant part, plant cell and/or plant genome may also be a plant, plant seed, plant part, plant cell and/or plant genome having a similar (but not the same or identical) genetic background to a modified plant, plant seed, plant part, plant cell and/or plant genome, if deemed sufficiently similar for comparison of the characteristics or traits to be analyzed.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. See, e.g., Khandagale, K. et al. (2016) “Genome editing for targeted improvement in plants,” Plant Biotechnol Rep 10: 327-343; and Gaj, T. et al. (2013) “ZFN, TALEN and CRISPR/Cas-based methods for genome engineering,” Trends Biotechnol. 31(7): 397-405. As used herein, “editing” or “genome editing” refers to generating a targeted mutation, deletion, inversion or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” also encompasses the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution or insertion, whereas “edits” or “genomic edits” refers to two or more targeted mutation(s), deletion(s), inversion(s), substitution(s) and/or insertion(s), with each “edit” being introduced via a targeted genome editing technique.

Targeted editing in the genome of a plant can be made by introducing a double strand break (DSB) or nick. According to this approach, mutations, such as deletions, insertions, inversions and/or substitutions may be introduced at a target site via imperfect repair of the DSB or nick to produce a knock-out or knock-down of an endogenous gene (i.e., a FAD gene). Such mutations may be generated by imperfect repair of the targeted locus even without the use of a donor template molecule. The DSB may be repaired via a Non-Homologous End Joining (NHEJ) pathway in the absence of any additional composition, via template-directed repair in the presence of a polynucleotide modification template, or via homologous recombination with a heterologous polynucleotide (donor DNA molecule). The HDR pathway repairs double-stranded DNA breaks and includes homologous recombination (HR) and single-strand annealing (SSA) (Lieber (2010) Annu. Rev. Biochem. 79:181-211).

A “knock-out” of a target gene, such as a FAD gene, may be achieved by inducing a DSB or nick at or near the endogenous locus of the target gene that results in non-expression of the protein or expression of a non-functional protein encoded by the target gene, whereas a “knock-down” of a gene may be achieved in a similar manner by inducing a DSB or nick at or near the endogenous locus of the target gene (e.g., a FAD gene) that is repaired imperfectly and reduces its expression but does not eliminate function of the encoded protein. For example, the site of the DSB or nick within the endogenous locus may be in the upstream or 5′ region of a targeted FAD gene (e.g., a promoter and/or enhancer sequence) to affect or reduce its level of expression. Similarly, such targeted knock-out or knock-down mutations of a FAD gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knock-out mutations of a FAD gene may be achieved by deleting or inverting at least a portion of the gene or by introducing a frame shift or premature stop codon into the coding sequence of the gene. A deletion of a portion of a FAD gene may also be introduced by generating DSBs or nicks at two target sites and causing a deletion of the intervening target region flanked by the target sites.

In some aspects, the genome editing techniques described herein can combine the introduction of a DSB with the introduction of an “exogenous” donor DNA molecule to produce a “knock-in” at a target site. An “exogenous” donor molecule, donor template, or donor template molecule (collectively a donor template), is a molecule that is not native to a specified system (e.g., a germplasm, variety, and/or plant) with respect to a nucleotide sequence and/or genomic location (i.e., locus) for a polynucleotide. Exogenous or heterologous polynucleotides or polypeptides may be molecules that have been artificially supplied to a biological system (e.g., a plant cell, a plant gene, a particular plant species or variety, and/or a plant chromosome) and are not native to that particular biological system. Thus, the designation of a nucleic acid as “exogenous” may indicate that the nucleic acid originated from a source other than a naturally-occurring source. Site-specific integration of an exogenous nucleic acid at a FAD locus may be accomplished by any technique known to those of skill in the art.

An exogenous donor template may include, for example, an insecticidal resistance gene, an herbicide tolerance gene, a nitrogen use efficiency gene, a water use efficiency gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system.

In one aspect, the present disclosure provides a method of targeted genome editing that comprises use of a CRISPR/Cas9 system. The CRISPR (clustered regularly interspaced short palindromic repeats) locus, which encodes RNA components of the system, and the cas (CRISPR-associated) locus, which encodes proteins (Jansen et al. (2002) Mal. Microbial. 43: 1565-1575; Makarova et al. (2002) Nucleic Acids Res. 30: 482-496; Makarova et al. (2006) Biol. Direct 1: 7; Haft et al. (2005) PLoS Comput. Biol. 1:e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H-N-H) nuclease domain, each of which can cleave a single DNA strand at a target sequence (the concerted action of both domains leads to DNA double strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al. (2014) Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

For RNA-guided endonucleases, a guide RNA (gRNA) molecule is further provided to direct the endonuclease to a target site in the genome of the plant via base-pairing or hybridization to cause a DSB or nick at or near the target site. The gRNA may be transformed or introduced into a plant cell or tissue (perhaps along with a nuclease, or nuclease-encoding DNA molecule, construct or vector) as a gRNA molecule, or as a recombinant DNA molecule, construct or vector comprising a transcribable DNA sequence encoding the guide RNA operably linked to a plant-expressible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is a RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as at or near a FAD gene. A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu, X. et al. (2014) “Target specificity of the CRISPR-Cas9 system,” Quant Biol. 2(2): 59-70. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

Genome editing using DSB-inducing agents, such as Cas endonuclease-gRNA complexes, has been described, for example in U.S. Patent Application US 2015-0082478 A1, published on Mar. 19, 2015, WO201 5/026886 A1, published on Feb. 26, 2015, WO2016007347, published on Jan. 14, 2016, and WO201625131, published on Feb. 18, 2016.

In one aspect of the disclosure, a sunflower FAD2-1 gene is edited via a genome editing technique. For genome editing at or near the FAD2-1 gene with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO:1 or a sequence complementary thereto. As used herein, the term “consecutive” in reference to a polynucleotide or protein sequence means without deletions or gaps in the sequence.

In one aspect, the disclosure provides a method for introducing knock-down mutations in sunflower by genome editing. An RNA-guided endonuclease may be targeted to an upstream or downstream sequence, such as a promoter and/or enhancer sequence, or an intron, 5′UTR, and/or 3′UTR sequence of a target gene, e.g., a FAD2-1 gene, to mutate one or more promoter and/or regulatory sequences of the gene and affect or reduce its level of expression.

In another aspect, the disclosure provides a method for introducing knock-out (and possibly knock-down) mutations in sunflower by genome editing. An RNA-guided endonuclease may be targeted to a coding and/or intron sequence of a FAD2-1 gene to eliminate expression and/or activity of a functional protein, e.g., a FAD2-1 protein, from the gene. A knock-out of a sunflower target (e,g, a FAD) gene, expression can also be achieved by targeting the upstream and/or 5′ UTR sequence(s) of the gene, or other sequences at or near the genomic locus of the target gene. Thus, a knock-out of FAD gene expression may be achieved by targeting a genomic sequence at or near the site or locus of sunflower FAD2-1 gene, an upstream or downstream sequence, such as a promoter and/or enhancer sequence, or an intron, 5′ UTR, and/or 3′ UTR sequence, of the FAD2-1 gene, which thereby eliminates or reduces the target gene expression.

According to some embodiments, guide RNAs for targeting the endogenous FAD2-1 gene are provided, which may comprise a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, or at least 21 consecutive nucleotides of SEQ ID NO:1. In further embodiments, guide RNAs for targeting an endogenous FAD2-1 gene are provided, which may comprise a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, or at least 20 consecutive nucleotides of SEQ ID NOs:2-3.

In addition to the guide sequence, a guide RNA may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

According to some aspects, a recombinant DNA construct or vector may comprise a first polynucleotide sequence encoding a site-specific nuclease and a second polynucleotide sequence encoding a guide RNA that may be introduced into a plant cell together via plant transformation techniques. In an aspect, vectors comprising polynucleotides encoding a site-specific nuclease, and optionally one or more, or two or more gRNAs are provided to a plant cell by transformation methods known in the art (e.g., without being limiting, particle bombardment, PEG-mediated protoplast transfection or Agrobacterium-mediated transformation). In an aspect, vectors comprising polynucleotides encoding a Cas9 nuclease, and optionally one or more, or two or more gRNAs are provided to a plant cell by transformation methods known in the art (e.g., without being limiting, particle bombardment, PEG-mediated protoplast transfection or Agrobacterium-mediated transformation). Methods for the introduction of Cas endonucleases and guide polynucleotide into plant cells are described, for example, in US 2016/0208272 A1, published 21 Jul. 2016, and in US 2016/0201072 A1, published 14 Jul. 2016. In the case of a CRISPR-Cas system, uptake of the endonuclease and/or the guided polynucleotide into the cell can be facilitated with a Cell Penetrating Peptide (CPP) as described in WO2016073433 published May 12, 2016.

Provided are recombinant DNA constructs or recombinant expression constructs which contain the sequences disclosed herein, including any combination of sequence components disclosed in the Examples. The term “recombinant DNA construct” or “recombinant expression construct” is used interchangeably and generally refers to a discrete polynucleotide into which a nucleic acid sequence or fragment can be moved. Preferably, it is a plasmid vector or a fragment thereof comprising the site-specific nuclease and polynucleotide sequences encoding the guide RNAs of the present disclosure, wherein the guide RNAs comprise guide sequences of sufficient length having a percent identity or complementarity to a target site within the genome of a plant, such as at or near a targeted FAD2-1 gene. According to some embodiments, a polynucleotide sequence of a recombinant DNA construct and vector that encodes a site-specific nuclease or a guide RNA may be operably linked to a plant expressible promoter, such as an inducible promoter, a constitutive promoter, a tissue-specific promoter, etc.

The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al. (1985) EMBO J. 4:241 1-2418; De Almeida et al. (1989) Mol. Gen. Genetics 218:78-86), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

Methods for transforming dicots, primarily by use of Agrobacterium tumefaciens, and obtaining transgenic plants have been published, among others, for cotton (U.S. Pat. Nos. 5,004,863, 5,159,135); soybean (U.S. Pat. Nos. 5,569,834, 5,416,011); Brassica (U.S. Pat. No. 5,463,174); sunflower (Schrammeijer et al. (1990) Plant Cell Reports, 9: 55-60, Weber et al. (2003) Plant Cell Rep. 21:475-482); papaya (Ling et al. (1991) Bio/technology 9:752-758); and pea (Grant et al. (1995) Plant Cell Rep. 15:254-258). For a review of other commonly used methods of plant transformation see Newell, C.A. (2000) Mol. Biotechnol. 16:53-65.

In another aspect, the genome editing techniques described can be used to provide a modified sunflower plant, or plant part thereof, comprising a heterozygous or homozygous mutant FAD2-1 gene. For example, a heterozygous or homozygous mutant FAD2-1 gene can include a mutation of its promoter, 5′ UTR, exon, intron, 3′ UTR, terminator, or any combination thereof. In another example, a heterozygous or homozygous mutant FAD2-1 gene can include a nonsense mutation, a missense mutation, a frameshift mutation, a splice-site mutation, or any combination thereof. A homozygous mutant FAD2-1 gene can result in one or more of the following: a protein truncation, a non-translatable transcript, a non-functional protein, a premature stop codon, and any combination thereof. The foregoing mutant FAD2-1 genes can comprise a substitution, a deletion, an insertion, a duplication, or an inversion of one or more nucleotides relative to a wild-type FAD2-1 gene. In certain examples, a mutant FAD2-1 gene comprises a null allele.

In another aspect, the genome editing techniques described can be used to make a modified sunflower plant that has a modified fatty acid profile. The modified fatty acid profile can be an increase in oleic acid as compared to the seeds of an unmodified control plant. The disclosed genome editing techniques described herein can modify the fatty acid profile of the modified sunflower plant without detrimentally affecting the value of the modified plant. In some cases, the value of the modified sunflower plant actually increases due to its modified fatty acid profile.

In an aspect, a modified sunflower plant is an inbred. In another aspect, a modified sunflower plant is a hybrid. In an aspect, a modified sunflower plant is a plant modified by a targeted genome editing technique.

For purposes of the present disclosure, a “plant” includes an explant, plant part, seedling, plantlet or whole plant at any stage of regeneration or development. As used herein, a “plant part” may refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed (e.g., embryo, endosperm, and seed coat), fruit (e.g., the mature ovary), propagule, or other plant tissues (e.g., vascular tissue, dermal tissue, ground tissue, and the like), or any portion thereof. Plant parts of the present disclosure may be viable, nonviable, regenerable, and/or non-regenerable. A “propagule” may include any plant part that can grow into an entire plant.

Sunflower plants that have been subjected to genome editing treatment may be screened and selected based on an observable phenotype, or using a selection agent (e.g., herbicide, etc.) to select for edits that introduce a selectable marker. Genome edited sunflower plants may be screened and selected using a screenable marker or a molecular phenotype (e.g., lower oleic acid levels, lower FAD2-1 transcript or protein levels, presence of transgene or transcribable sequence, and the like).

In some aspects, the disclosed method can include detecting modified nucleic acids and/or polypeptides in plant cells. For example, modified nucleic acids may be detected using hybridization probes or through production of amplicons using the polymerase chain reaction (PCR) with primers as known in the art. A “probe” is generally referred to an isolated/synthesized nucleic acid, either DNA or RNA, and may be prepared synthetically or by cloning. Suitable cloning vectors are well known to those skilled in the art. RNA probes can be synthesized by means known in the art, for example, using a DNA molecule template. “Primers” generally referred to isolated/synthesized nucleic acids that hybridize to a complementary target DNA strand which is then extended along the target DNA strand by a polymerase, e.g., a DNA polymerase. Primer pairs often used for amplification of a target nucleic acid sequence, e.g., by PCR or other conventional nucleic-acid amplification methods. Primers are also used for a variety of sequencing reactions, sequence captures, and other sequence-based amplification methodologies. Such probes and primers are used in hybridization reactions to target DNA or RNA sequences under high stringency hybridization conditions or under lower stringency conditions, depending on the need. Hybridization between nucleic acids is discussed in Sambrook et al. (1989, Molecular Cloning: A Laboratory Manual, 2nd Ed., Cold Spring Harbor Laboratory Press, Cold Spring Harbor, N.Y.).

Modified polypeptides and/or modified levels of polypeptides can be detected using antibodies. Techniques for detecting polypeptides using antibodies include enzyme linked immunosorbent assays (ELISAs), Western blots, immunoprecipitations, immunofluorescence, and the like. An antibody provided herein may be a polyclonal antibody or a monoclonal antibody. An antibody having specific binding affinity for a polypeptide provided herein can be generated using methods known in the art. An antibody or hybridization probe may be attached to a solid support, such as a tube, plate or well, using methods known in the art.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials.

Techniques that can be used in screening and selection of modified or edited plants or plant cells can include any methodologies known in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides.

The disclosure provides a method of detecting the presence of a modification in a FAD2-1 allele (SEQ ID NO:1). The method includes providing a plurality of samples comprising sunflower genomic DNA, contacting the sample with a pair of DNA primers and two probes, and then performing a nucleic acid amplification reaction to generate an amplicon. In a first example, the pair of primers includes a first and second primer comprising SEQ ID NO:6 and SEQ ID NO:7, respectively, such that detection of an amplicon using a wild-type probe comprising SEQ ID NO: 8 indicates the presence of a wild-type sequence in the CR3 target site of the FAD2-1 gene, while a decreased detection signal or absence of a detection signal indicates a disrupted or modified CR3 target site in the FAD2-1 gene. A decreased detection signal by the wild-type probe (SEQ ID NO: 8) at the disrupted target site is determined when compared to the detection signal of a CR3 standard probe (SEQ ID NO:12) which is designed downstream of the CR3 target site. In a second example, the first and second primers comprise SEQ ID NO:9 and SEQ ID NO:10, respectively, such that detection of an amplicon using a wild-type probe comprising SEQ ID NO:11 indicates the presence of a wild-type sequence in the CR4 target site of the FAD2-1 gene, while a decreased detection signal or absence of a detection signal indicates a disrupted or modified CR4 target site in the FAD2-1 gene. A decreased detection signal by the wild-type probe (SEQ ID NO:11) at the disrupted target site is determined when compared to the detection signal of a CR4 standard probe (SEQ ID NO:13) which is designed upstream of the CR4 target site. Each of the probes described herein can be labeled to facilitate detection. For example, a probe can be radiolabeled (e.g., with P³² or S³⁵) or fluorescently labeled. Examples of fluorescent labels include a HEX fluorescent dye, a VIC fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, or a ROX fluorescent dye.

In some aspects, the genome editing techniques described herein may provide a modified sunflower plant having a significantly reduced or eliminated expression level of the FAD2-1 gene transcript and/or protein in one or more tissue(s) of the modified plants, as compared to the same tissue(s) of wild-type or control plants. Such screening and/or selecting techniques may be used to identify and select plants having a mutation in a FAD2-1 gene that leads to a desirable plant phenotype. In other aspects, the genome editing techniques described herein may provide a modified sunflower plant that has a modified fatty acid profile. In another aspect, the modified fatty acid profile, comprises an increase in oleic acid in the sunflower seeds compared to the seeds of an unmodified control plant, that is measured using standard protocols known in the art (e.g., Fatty Acid Methyl Ester GC).

### EXAMPLES

Disclosed herein are examples of genome editing in sunflower. Sunflower FAD2-1 locus was used as a target site to demonstrate targeted genome editing in two different transformable sunflower genotypes (Line F and Line N). Agrobacterium-mediated transformation was used to deliver CRISPR-Cas9 reagents. Next Generation Sequencing (NGS) analysis of DNA extracted from these plants revealed up to 88% mutation frequency at one of the target sites. PCR analysis indicated low-frequency potentially chimeric dropout in some T0 plants. Three T0 plants detected positive for edits at one or both target sites were moved to maturity and seed production. T1 analysis showed stable inheritance of FAD2 frameshift edits. T1 progeny containing targeted edits with segregation of CRISPR-Cas9 reagents were observed from one plant.

### Example 1: Target Site Selection, Guide Design and Vector Construction

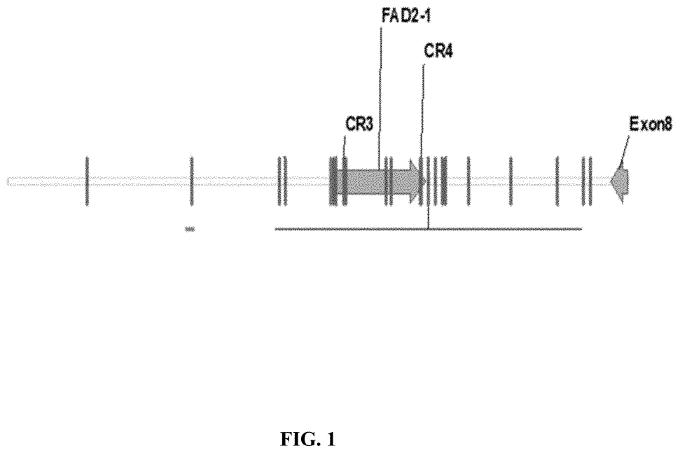

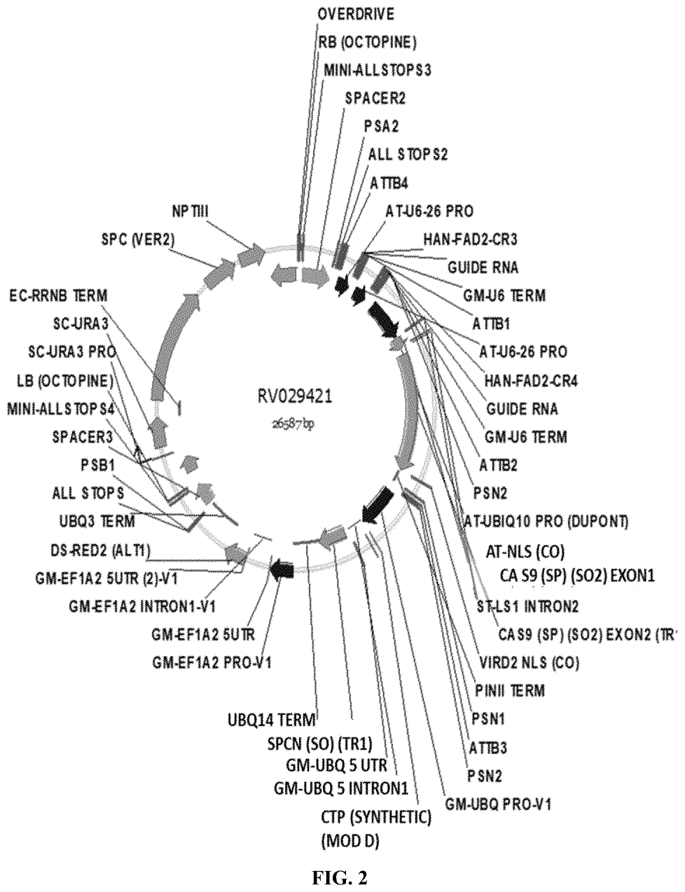

Guide RNAs were designed to create targeted frameshift mutations and full-length gene dropout at the sunflower FAD-2 locus. Helianthus annuus FAD2-1 sequence (SEQ ID NO:1) is shown in FIG. 1. Two guide RNA sequences, CR3 (SEQ ID NO:2) and CR4 (SEQ ID NO:3) target two different sites in the coding region of FAD2-1 and were built into the vector RV029421 described in FIG. 2. Each of the guide RNA sequences is driven by an independent Arabidopsis U6 (AT-U6-26 PRO; SEQ ID NO:4) promoter. Arabidopsis UBI10 (AT-UBIQ10 PRO; SEQ ID NO:5) promoter was used to express Cas9. Spectinomycin was used as selectable transformation marker. In addition, DsRed was used as a visible transformation marker. The plant transformation vector RV029421 was mobilized into LBA4404 (TD-THY) Agrobacterium strain containing secondary plasmid PHP71539.

### Example 2: Embryo Axis Preparation for Transformation

50 grams of sunflower seed were weighed for dehulling. The seed was briefly washed in 70% ethanol for 3-5 minutes, decanted and left to air dry. Seed was then processed in Santec VILI11 Laboratory Sunflower and Spelt Huller, (Evol consulting S.R.O., Vydrany, Slovakia) until most seed had been shelled. Processed seed was hand sorted to remove embryos that were released from the shell. Unshelled seed was further processed in the dehuller until most seed was dehulled. Embryos were then washed in a solution containing sterile de-ionized water and about 2% PPM (Plant Preservative Mixture, Plant Cell Technology, Washington, D.C.). The wash solution was prepared and used at a ratio of 4:1 v/w. Embryos were agitated at room temp on a shaker table set to “4” (Orbital Shaker, Bellco, Vineland, N.J.) in enough volume to cover the embryos and to allow movement of embryos while being agitated. Washing solution was replaced with fresh solution after 2-3 hours, and shaking continued overnight.

After the dehulling, washing solution was decanted and embryo axes (EAs) were rinsed 3-5 times with sterile deionized water with agitation until the tegmans are removed from the embryos. After rinsing, EAs were isolated from the embryos and prepared for infection following a modified version of the protocol described by Schrammeijer et al. (1990) Meristem transformation of sunflower via Agrobacterium. Plant Cell Rep 9, 55-60. The cotyledons and leaf primordia were removed with forceps and a #11 scalpel blade. For embryos where the tegmen had been washed off, the cotyledons were separated from each other to permit one cotyledon to be broken off with forceps and then the plumule and second cotyledon were cut off together, exposing the apical dome of the meristem and leaving behind just the embryo axis. When tegmen was still attached to the embryo it was peeled away and then the cotyledons would be cut at the point where they attach to the radicle. If the plumule was still present, it too was cut away. Isolated EAs were transferred to infection media until ready for infection.

### Example 3: Agrobacterium Preparation and Infection of EAs

The day before EA preparation, Agrobacterium tumefaciens strain RV029421 (LBA4404 thy-) was struck from a colony growing on a master plate of 12R media (Table 1) stored at 4° C. onto a working plate of 810K media (Table 2) and incubated overnight at 27° C. The cultured Agrobacterium was then added to 620e infection media (Table 3) supplemented with 100 μM dithiothreitol (DTT) and 200 μM acetosyringone (AS) and adjusted to an OD of 0.50 @ 550 nM. The Agrobacterium solution was adjusted to 25 mL of suspension and 50 μl Silwett was added. Twelve mL of the suspension was added to the EAs in a 100×25 mm petri plate and sonicated for 30 seconds. After sonication, the remaining 13 mL of Agrobacterium suspension was added and EAs were placed under vacuum for 30 minutes. After vacuum infiltration, the EAs were placed into neat piles on filter paper in a fresh 100×25 mm plate that had been moistened with 700 mL of the Agrobacterium suspension. The EAs were incubated for three days under dim light (10-13 μmol/s2) and 24° C.

Co-culture of EAs was terminated by embedding them vertically into selection media 15720K (Table 4) and placed in 26° C. culture room. After two to three weeks, spectinomycin resistant/RFP positive sectors could be identified. Shoots containing multiple leaves were transferred to 90 media (Table 5) for rooting once a proper shoot had formed. Once roots were developed, rooted, and plants reached a height of 2-3 cm, they were sent to the greenhouse for further growth.

### Example 4: Confirmation of Gene Editing by qPCR and NGS

Eight T0 plants regenerated on selection media were analyzed for frameshift mutations at CR3/CR4 sites and dropout of the FAD2-1 sequence (FIG. 1). Multiple samples were taken from different leaves of each plant to evaluate potential chimerism. PCR across the CR3 (SEQ ID NO:2) and CR4 (SEQ ID NO:3) target sites (FIG. 1) was performed to detect gene dropout using the CR3 forward primer (SEQ ID NO:6) and the CR4 reverse primer (SEQ ID NO:10). A strong 1.1 kb PCR product indicated a wild-type sequence and a faint 161 bp product indicated a dropout. PCR results of T0 plants are shown in Table 7. The frameshift mutations were analyzed both by quantitative PCR (qPCR) and Next Generation Sequencing (NGS). To identify mutations at the CR3 target site, qPCR was performed with CR3 forward and reverse primers SEQ ID NO:6 and SEQ ID NO:7, respectively, and CR3 wild-type probe (SEQ ID NO:8) and CR3 standard probe (SEQ ID NO:12). Detection of an amplicon indicated the presence of a wild-type sequence in the CR3 target site of the FAD2-1 gene, while a decreased detection signal or absence of a detection signal indicated a disrupted or modified CR3 target site in the FAD2-1 gene. A decreased detection signal by the wild-type probe (SEQ ID NO: 8) at the disrupted target site was determined when compared to the detection signal of the CR3 standard probe (SEQ ID NO:12) which is designed downstream of the CR3 target site. To identify mutations at the CR4 target site, qPCR was performed with CR4 forward and reverse primers SEQ ID NO:9 and SEQ ID NO:10, respectively, and CR4 wild-type probe (SEQ ID NO:11) and CR4 standard probe (SEQ ID NO:13). Standard methods known in the art were used for DNA extraction, PCR, qPCR and NGS sequencing. Primers and probes are listed in Table 6.

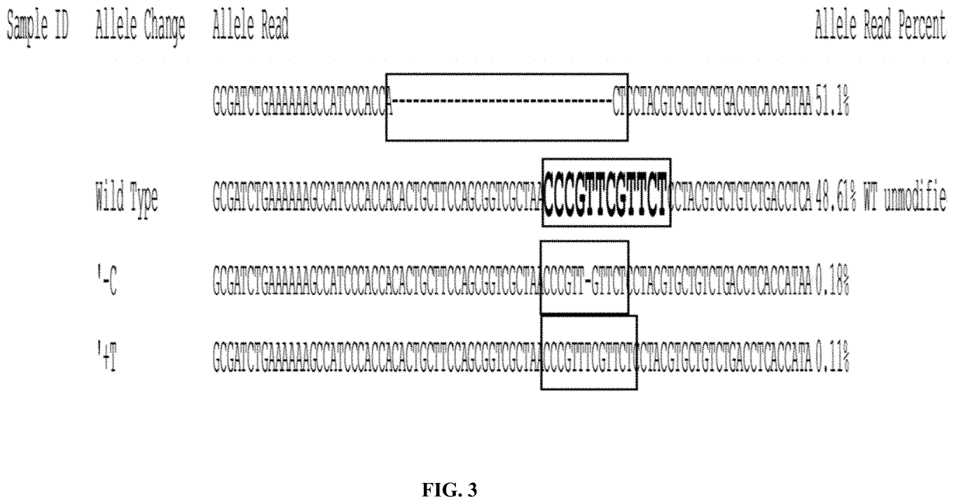

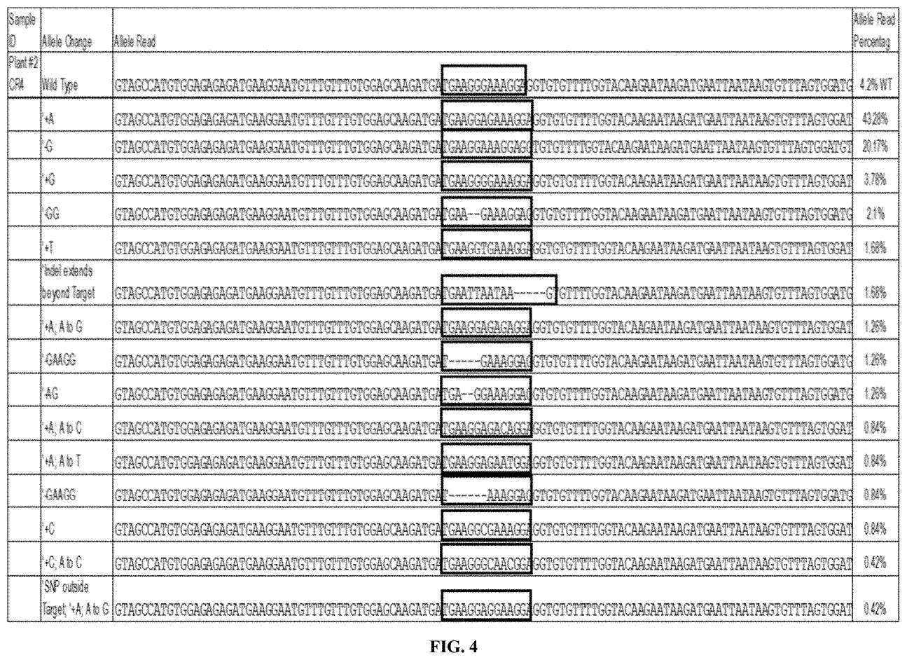

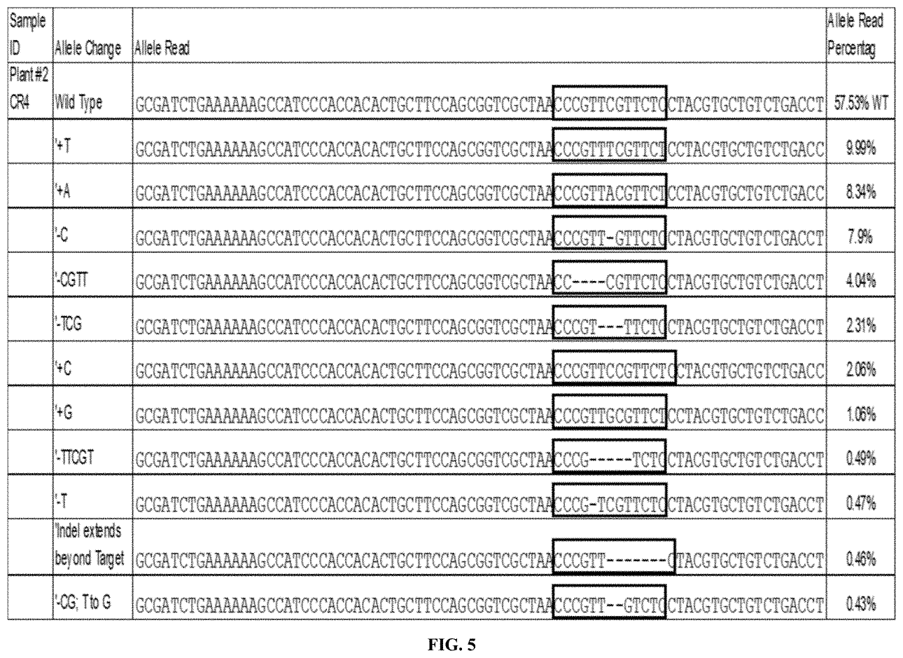

Approximately 50% of the NGS reads from all the samples derived from Plant 1 (Line F genotype) were observed to contain mutations at CR3 site. Illustrative sequence modifications are shown in FIG. 3. No mutation at the CR4 site or dropout was observed in this plant (Table 7). The qPCR mutation at CR3 was not detected because of a small deletion outside of the probe binding site. The remaining T0 plants were regenerated from the genotype Line N. Mutations equivalent to one allele (˜60% NGS reads, <1 qPCR) for the CR4 site were observed from all the samples analyzed from Plant 2. Illustrative sequence modifications are shown in FIG. 4. A low level of mutation at the CR3 site and chimeric dropout were also observed in this plant (Table 7, FIG. 5). Unlike Plants 1 and 2, Plants 3-8 were observed to be chimeric both for mutations and dropout. One sample of Plant 3 showed mono- and bi-allelic mutations for the site CR3 and CR4, respectively. No mutation was observed from the second sample analyzed from the same plant.

To study inheritance of edits made in T0 plants, three T0 plants (Plants 1, 2, and 7) were taken to maturity and self-fertilized for seed production. T1 progeny were analyzed for CR3/CR4 mutations and dropout. Expected inheritance of CR3 and CR4 mutations in the progeny of Plants 1 and 2, respectively were observed (Table 8, see summary of T1 data in Table 9). No mutation was observed in the progeny of Plant 7, which was detected chimeric in T0 stage. Progeny of Plant 1 contained multiple copies of Cas9 reagents and therefore no plant was observed without Cas9 reagents. In contrast, two T1 plants from the T0 Plant 2 contained the CR4 mutation and were PCR negative for Cas9 reagents (Table 9).

### Example 5: Compositional Analysis of the Sunflower Seeds of Edited Events

The saturated fatty acid content of sunflower seeds of edited events is determined by standard Fatty Acid Methyl Ester (FAME) GC procedures wherein the oil is removed from the seeds by crushing the seeds and is extracted as fatty acid methyl esters following reaction with methanol and sodium hydroxide. The resulting ester is analyzed for fatty acid content by gas liquid chromatography using a capillary column which allows separation on the basis of the degree of unsaturation and chain length. See, for example, J. K. Daun et al. (1983) J. Amer. Oil Chem. Soc. 60: 1751-1754. The genome editing mutation of FAD2-1 gene leads to an increase in oleic acid in the sunflower seeds.

TABLE 1
Media 12R

| | Ingredient | I | | M |
|---|---|---|---|---|
| Ingredient | Amount | units | Molarity | units |
| GLUCOSE | 5 | g/L | 27.747 | mM |
| Bacto Agar | 15 | g/L | | |
| 12B-AB FeSO4•7H20 | 2 | mL/L | | |
| STOCK 5X | | | | |
| 12D-AB BUFFER 20X | 50 | mL/L | | |
| 12E-AB SALTS 20X | 50 | mL/L | | |
| 47-SPECTINOMYCIN | 1 | mL/L | 100.939 | μM |
| DIHYDROCHLORIDE | | | | |
| 50 MG/ML | | | | |
| 14020-Thymidine | 1 | mL/L | 206.415 | μM |
| 50 mg/mL | | | | |
| 17K-Gentamicin | 1 | mL/L | 86.855 | μM |
| 50 mg/mL | | | | |

TABLE 2
Media 810K

| | Ingredient | I | | M |
|---|---|---|---|---|
| Ingredient | Amount | units | Molarity | units |
| YEAST EXTRACT | 5 | g/L | | |
| (BD Difco) | | | | |
| PEPTONE | 10 | g/L | | |
| Sodium Chloride | 5 | g/L | 85.558 | mM |
| Bacto Agar | 15 | g/L | | |
| 47-SPECTINOMYCIN | 1 | mL/L | 100.939 | μM |
| DIHYDROCHLORIDE | | | | |
| 50 MG/ML | | | | |
| 14020-Thymidine | 1 | mL/L | 206.415 | μM |
| 50 mg/mL | | | | |
| 17K-Gentamicin | 1 | mL/L | 86.855 | μM |
| 50 mg/mL | | | | |

TABLE 3
Media 620E

| | Ingredient | I | | M |
|---|---|---|---|---|
| Ingredient | Amount | units | Molarity | units |
| GAMBORG B5 BASAL | 0.321 | g/L | | |
| MEDIUM (G398) | | | | |
| Sucrose | 30 | g/L | 87.632 | mM |
| MES BUFFER | 4.26 | g/L | | |
| 13108-BAP 1 MG/ML | 1.67 | mL/L | | |
| (13108BASE VAX 14I) | | | | |
| 17G-Gibberellic | 2.5 | mL/L | 288.675 | nM |
| Acid 0.1 mg/mL | | | | |

TABLE 4
Media 1570K

| | Ingredient | I | | M |
|---|---|---|---|---|
| Ingredient | Amount | units | Molarity | units |
| Rugini Olive Medium Mix | 4.05 | g/L | | |
| Sucrose | 40 | g/L | 116.843 | mM |
| Bacto Agar | 6 | g/L | | |
| 17F-MEROPENEM | 2 | mL/L | 11.428 | μM |
| 5 mg/ml | | | | |
| 14081-Meta-Topolin | 0.2 | mL/L | 2.07 | μM |
| 0.5 mg/mL | | | | |
| 47-SPECTINOMYCIN | 0.8 | mL/L | 100.939 | μM |
| DIHYDROCHLORIDE | | | | |
| 50 MG/ML | | | | |

TABLE 5
Media 90

| | Ingredient | I | | M |
|---|---|---|---|---|
| Ingredient | Amount | units | Molarity | units |
| MS BASAL SALT | 2.165 | g/L | | |
| MIXTURE | | | | |
| Sucrose | 20 | g/L | 58.421 | mM |
| SIGMA AGAR | 8 | g/L | | |

TABLE 6
Oligonucleotides for qPCR and NGS sequencing

| SEQ | | |
|---|---|---|
| ID | | |
| NO: | Oligo | Sequence 5′ - 3′ |
| 6 | CR3-Forward Primer | GCGATCTGAAAAAAGCCATCC |
| 7 | CR3-Reverse Primer | GGTAGAGGACAGCGGTTATGGT |
| 8 | CR3 Wild-type Probe | ACCCGTTCGTTCTCCTA |
| 9 | CR4-Forward Primer | TGTAGCCATGTGGAGAGAGATGA |
| 10 | CR4-Reverse Primer | AGACCCAGAACCAGGACAAGAC |
| 11 | CR4 Wild-type Probe | ACCTCCTTTCCCTTCAT |
| 12 | CR3 Standard Probe | ACCACACTGCTTCCA |
| 13 | CR4 Standard Probe | AGTGGATGTCTATGCTTAT |

The same primers were used for both qPCR and NGS sequencing.

TABLE 7
FAD2.1 CR3 and CR4 mutations and dropout in T0 generation

| | | | qPCR* | | NGS^{Φ} | | |
|---|---|---|---|---|---|---|---|
| | | | mutation | | mutation | | |
| T0 Plant | Genotype | Sample | CR3 | CR4 | CR3 | CR4 | Dropout |
| 1 | Line F | 1 | ND | 2.01 | 52.52 | 0.86 | Neg |
| 1 | Line F | 2 | 1.88 | 1.98 | 51.39 | 0 | Neg |
| 1 | Line F | 3 | 1.91 | 1.78 | 52.31 | 0 | Neg |
| 1 | Line F | 4 | 1.83 | 1.87 | 51.73 | 0.45 | Neg |
| 1 | Line F | 5 | 1.87 | 1.69 | 52.43 | 0 | Neg |
| 1 | Line F | 6 | 1.86 | 1.76 | 52.51 | 0.51 | Neg |
| 2 | Line N | 1 | 1.77 | 0.68 | 9.01 | 63.04 | Weak Pos |
| 2 | Line N | 2 | 1.76 | 0.69 | 4.92 | 69.89 | Neg |
| 2 | Line N | 3 | 1.54 | 0.91 | 9.82 | 63.39 | Weak Pos |
| 3 | Line N | 1 | 1.12 | 0.16 | 42.48 | 95.79 | Neg |
| 3 | Line N | 2 | 1.84 | 1.48 | 0.1 | 0 | Neg |
| 4 | Line N | 1 | 1.22 | 0.24 | 34.93 | 88.35 | Weak Pos |
| 4 | Line N | 2 | 1.61 | 1.72 | 3.67 | 22 | Weak Pos |
| 5 | Line N | 1 | 1.7 | 1.45 | 9.83 | 29.4 | Weak Pos |
| 5 | Line N | 2 | 1.78 | 1.49 | 8.35 | 26.72 | Weak Pos |
| 6 | Line N | 1 | 1.85 | 1.71 | 0.64 | 10.54 | Neg |
| 7 | Line N | 1 | 1.86 | 0.96 | 5.16 | 48.6 | Weak Pos |
| 7 | Line N | 2 | 1.88 | 1.83 | 0.16 | 0.33 | Neg |
| 8 | Line N | 1 | 1.91 | 1.36 | 0.59 | 33.09 | Neg |
| Control | Line F | 1 | 1.88 | 1.85 | 0.14 | 0.49 | Neg |
| Control | Line F | 1 | 1.9 | 1.76 | 0.12 | 0.43 | Neg |
| Control | Line F | 1 | 2.01 | 1.77 | 0.22 | 0.4 | Neg |
| Control | Line F | 1 | 1.91 | 1.77 | 0.14 | 0.48 | Neg |
| Control | Line N | 1 | 1.9 | 1.56 | 0.18 | 0.7 | Neg |
| Control | Line N | 1 | 2.95 | 1.73 | 0.17 | 0.41 | Neg |
| Control | Line N | 1 | 1.8 | 1.76 | 0.12 | 0.56 | Neg |
| Control | Line N | 1 | 1.74 | 1.78 | ND | ND | Neg |

*WT = ~2
^{Φ}% reads with mutations
ND: Not determined
Pos: Positive
Neg: Negative

TABLE 8
FAD2.1 CR3 and CR4 mutations and dropout in T1 generation

| | | NGS^{ϕ} mutation | | |
|---|---|---|---|---|
| Genotype | T0 Plant | CR3 | CR4 | CAS9 qPCR |
| Line F | 1 | 52.6 | WT | 14.5 |
| Line F | 1 | 56.1 | WT | 8.2 |
| Line F | 1 | 56.4 | WT | 6.3 |
| Line F | 1 | 56.5 | WT | 13.7 |
| Line F | 1 | 56.7 | WT | 13.9 |
| Line F | 1 | 57.1 | WT | 14.2 |
| Line F | 1 | 57.5 | WT | 11.1 |
| Line F | 1 | 57.6 | WT | 5.9 |
| Line F | 1 | 58 | WT | 14.2 |
| Line F | 1 | 58 | WT | 17.6 |
| Line F | 1 | 58.1 | WT | 6.1 |
| Line F | 1 | 58.3 | WT | 12.1 |
| Line F | 1 | 58.3 | WT | 17.8 |
| Line F | 1 | 58.3 | WT | 10.5 |
| Line F | 1 | 58.4 | WT | 10.6 |
| Line F | 1 | 58.4 | WT | 16.1 |
| Line F | 1 | 58.5 | WT | 14.1 |
| Line F | 1 | 59.1 | WT | 14.1 |
| Line F | 1 | 59.2 | WT | 5.8 |
| Line F | 1 | 59.2 | WT | 18.8 |
| Line F | 1 | 59.2 | WT | 15 |
| Line F | 1 | 59.4 | WT | ND |
| Line F | 1 | 59.5 | WT | 12.7 |
| Line F | 1 | 59.7 | WT | 12.1 |
| Line F | 1 | 99.8 | WT | 8.3 |
| Line F | 1 | 99.8 | WT | 9.4 |
| Line F | 1 | 99.8 | WT | 7.2 |
| Line F | 1 | 99.8 | WT | 13.9 |
| Line F | 1 | 99.9 | WT | 8 |
| Line F | 1 | 99.9 | WT | 14 |
| Line F | 1 | 99.9 | WT | 9.3 |
| Line F | 1 | 99.9 | WT | 12.3 |
| Line F | 1 | 99.9 | WT | 8.8 |
| Line F | 1 | 99.9 | WT | 8.3 |
| Line F | 1 | 99.9 | WT | 9.3 |
| Line F | 1 | 99.9 | WT | 9 |
| Line F | 1 | 100 | WT | 10.5 |
| Line F | 1 | 100 | WT | 13.6 |
| Line F | 1 | 100 | WT | 12.7 |
| Line F | 1 | 100 | WT | 8.2 |
| Line F | 1 | WT | WT | 10.6 |
| Line F | 1 | WT | WT | 12.4 |
| Line F | 1 | WT | WT | 18.5 |
| Line F | 1 | WT | WT | 11.6 |
| Line F | 1 | WT | WT | 13.4 |
| Line F | 1 | WT | WT | 13.4 |
| Line F | 1 | WT | WT | 6.4 |
| Line F | 1 | WT | WT | 7.7 |
| Line N | 2 | 0.1 | 52.1 | null |
| Line N | 2 | 0.3 | 52.2 | null |
| Line N | 2 | 10.9 | 65 | 0.8 |
| Line N | 2 | 8.4 | 66.9 | 0.9 |
| Line N | 2 | 12.6 | 82.2 | 0.9 |
| Line N | 2 | 56.6 | 82.6 | 0.9 |
| Line N | 2 | 57.8 | 83.5 | 0.9 |
| Line N | 2 | 10.6 | 84.7 | 1.3 |
| Line N | 2 | 11.6 | 85.5 | 0.9 |
| Line N | 2 | 9.6 | 87.1 | 0.9 |
| Line N | 2 | 10.5 | 89.4 | 1 |
| Line N | 2 | 14.5 | 91.6 | 0.8 |
| Line N | 2 | 17.2 | 93.7 | 1.2 |
| Line N | 2 | 12.7 | 94.1 | 0.9 |
| Line N | 2 | 17 | 95.2 | 0.9 |
| Line N | 2 | 18.3 | 95.5 | 1 |
| Line N | 2 | 43 | 97.7 | 1.8 |
| Line N | 2 | 43.3 | 98.9 | 1.8 |
| Line N | 2 | 12.9 | 99.9 | 0.9 |
| Line N | 2 | 13.7 | 99.9 | 1 |
| Line N | 2 | 14.4 | 99.9 | 0.7 |
| Line N | 2 | 12.2 | 99.9 | 0.8 |
| Line N | 2 | 24.1 | 99.9 | 2.2 |
| Line N | 2 | 28.7 | 99.9 | 2.2 |
| Line N | 2 | 10.9 | 99.9 | 0.9 |
| Line N | 2 | 35.3 | 99.9 | 1.6 |
| Line N | 2 | 42.3 | 99.9 | 2.1 |
| Line N | 2 | 8 | 99.9 | 0.8 |
| Line N | 2 | WT | WT | null |
| Line N | 2 | WT | WT | null |
| Line N | 2 | WT | WT | null |
| Line N | 2 | WT | WT | null |
| Line N | 2 | WT | WT | null |
| Line N | 2 | WT | WT | null |
| Line N | 2 | WT | WT | null |
| Line N | 2 | WT | WT | null |
| Line N | 7 | WT | WT | null |
| Line N | 7 | WT | WT | null |
| Line N | 7 | WT | WT | null |
| Line N | 7 | WT | WT | null |
| Line N | 7 | WT | WT | null |
| Line N | 7 | WT | WT | null |
| Line F | 7 | WT | WT | null |
| Line F | 7 | WT | WT | null |
| Line F | 7 | WT | WT | null |
| Line F | 7 | WT | WT | null |

^{ϕ}% reads with mutations
ND: Not determined

TABLE 9
Summary of FAD2.1 CR3 and CR4 mutations in T1 generation

| T0 Plant | Cut site | Total | Homo | Hemi | WT | Cas9 reagent free |
|---|---|---|---|---|---|---|
| 1 | CR3 | 48 | 16 | 24 | 8 | 0 |
| 2* | CR4 | 46 | 17 | 11 | 18 | 2 |

*>90% Homo, 50-90% Hemi, Number of mutant reads overestimated due to presence of Cas9 reagents

1. A method for modifying a target site in the genome of a sunflower plant cell, the method comprising:
providing a recombinant DNA construct comprising nucleic acid sequence encoding one or more guide RNAs to a sunflower plant cell having a Cas endonuclease;
expressing the one or more guide RNAs to form a complex with Cas endonuclease that enables the Cas endonuclease to introduce a double strand break at a target site in the plant cell's genome, wherein the double strand break at said target site is repaired via an imperfect repair that introduces a targeted modification at the target site.

2. (canceled)

3. The method of claim 1, wherein the targeted modification includes at least one insertion, deletion or substitution of one or more nucleotides in said target site.

4. The method of claim 1, wherein the introduction of the nucleotide modification in the target site results in one or more of the following: reduced expression of a polynucleotide encoding a polypeptide; reduced activity of a polypeptide; generation of one or more alternative spliced transcripts of a polynucleotide encoding a polypeptide; deletion of one or more active sites of the polypeptide; frameshift mutation in one or more exons of a polynucleotide encoding the polypeptide; deletion of a substantial portion or the full length of polynucleotide encoding a polypeptide; repression of an enhancer motif present within a regulatory region operably linked to a sequence encoding a polypeptide; or modification of one or more nucleotides of a regulatory element in a promoter, intron, 3′UTR, or terminator that is operably linked to the expression of the polynucleotide encoding the polypeptide.

5. The method of claim 1, wherein the targeted modification is in a FAD2 gene target site.

6. The method of claim 5, wherein the FAD2 gene nucleotide sequence is at least 90% identical to SEQ ID NO:1.

7. The method of claim 1, wherein the one or more guide RNAs comprise a first guide RNA comprising SEQ ID NO:2 and a second guide RNA comprising SEQ ID NO:3.

8. The method of claim 7, wherein the method further comprises generating a plant comprising the targeted modification at the target site.

9-14. (canceled)

15. The method of claim 3, wherein the method further includes delivering a nucleic acid sequence of interest to the sunflower plant cell, which is integrated into the double strand break at the target site.

16. The method of claim 15, wherein the nucleic acid sequence of interest is selected from the group consisting of a fatty acid metabolism gene, carbohydrate metabolism gene, insecticidal resistance gene, herbicidal tolerance gene, nutritional quality gene, yield gene, pest or disease resistance gene, drought tolerance gene, stress tolerance gene, nitrogen use efficiency gene, or water use efficiency gene.

17. The method of claim 15, wherein the nucleic acid sequence of interest is integrated into a FAD2 gene.

18. The method of claim 17, wherein the FAD2 gene nucleotide sequence is at least 90% identical to SEQ ID NO:1.

19. The method of claim 15, wherein the one or more guide RNAs comprise a first guide RNA comprising SEQ ID NO:2 and a second guide RNA comprising SEQ ID NO:3.

20. A cell, seed or plant comprising a cell modified according to the method of claim 15.

21. The plant of claim 20, wherein the nucleic sequence of interest is stably incorporated into its genome, and wherein the plant is a sunflower plant.

22. (canceled)

23. A seed produced by the plant of claim 21.

24. A recombinant DNA construct encoding a first guide RNA comprising SEQ ID NO:2 and a second guide RNA comprising SEQ ID NO:3.

25. A cell, seed, or plant comprising the recombinant DNA construct of claim 24.

26. A plant having stably incorporated into its genome the recombinant DNA construct of claim 24, wherein the plant is a sunflower plant.

27. (canceled)

28. A seed produced by the plant of claim 26.

29. The seed of claim 28, wherein the nucleotide modification in the FAD2 gene results in an increase of oleic acid content compared to the seed of a control sunflower plant.


SEQUENCE LISTING (13 sequences)

SEQ ID NO: 1 - DNA, 1137 bp, Helianthus annuus
atgggtgcaggagaatacacgtctgtgaccaacgaaaacaacccactcgatcgagtccctcatgcaaaaccacccttcaccatcggcgatctgaaaaaagccatcccaccacactgcttccagcggtcgctaacccgttcgttctcctacgtgctgtctgacctcaccataaccgctgtcctctaccacattgccaccacctacttccaccacctccccacccctttgtcatccatcgcatgggcctcttactgggtagtccaaggctgcgtcctcaccggagtctgggtcatcgcccacgaatgtggtcaccatgcgtttagtgattatcaatgggtcgacgacactgtgggctttgttctccactcgtctttactcgtcccttacttttcgtggaaatatagtcaccaccgccaccattccaacactggatcactcgagcgggacgaggttttcgtccccaaatcccgatcgaaagtcccgtggtactcgaaatactttaacaacacagtgggccgcattgtcagtatgttcgtcactctcactctcggctggcccttgtacttagctttcaatgtgtcgggccgaccctatgaccgtttcgcctgccactacgtcccaaccagccctatgtacaatgaacgtaaacgttaccagatagtcatgtccgacatcgggattgttatcacatcgttcatcctttatcgtgttgctatggcaaaagggttggtttgggtgatttgcgtctatggggttccgttgatggttgtgaacgcgtttctggtgttgatcacttatcttcaacatactcaccctggcttgccgcattatgatagctcggaatgggaatggttaaagggagcattggcgacagtggaccgtgactatggtgtgttgaacaaggtgttccatcatattaccgacacacatgtggtgcaccatttgttttcgacaatgcctcattataatgcgatggaagcacagaaggcgctgagaccggtgcttggggagtattatcggtttgacaagaccccgttttatgtagccatgtggagagagatgaaggaatgtttgtttgtggagcaagatgatgaagggaaaggaggtgtgttttggtacaagaataagatgaattaa

SEQ ID NO: 2 - DNA, 20 bp, Helianthus annuus
gttcgttctcctacgtgctg

SEQ ID NO: 3 - DNA, 20 bp, Helianthus annuus
gagcaagatgatgaagggaa

SEQ ID NO: 4 - DNA, 445 bp, Arabidopsis thaliana
cttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 5 - DNA, 1305 bp, Arabidopsis thaliana
atcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaaca

SEQ ID NO: 6 - DNA, 21 bp, Artificial Sequence
  synthesized oligonucleotide
gcgatctgaaaaaagccatcc

SEQ ID NO: 7 - DNA, 22 bp, Artificial Sequence
  synthesized oligonucleotide
ggtagaggacagcggttatggt

SEQ ID NO: 8 - DNA, 17 bp, Artificial Sequence
  synthesized oligonucleotide
acccgttcgttctccta

SEQ ID NO: 9 - DNA, 23 bp, Artificial Sequence
  synthesized oligonucleotide
tgtagccatgtggagagagatga

SEQ ID NO: 10 - DNA, 22 bp, Artificial Sequence
  synthesized oligonucleotide
agacccagaaccaggacaagac

SEQ ID NO: 11 - DNA, 17 bp, Artificial Sequence
  synthesized oligonucleotide
acctcctttcccttcat

SEQ ID NO: 12 - DNA, 15 bp, Artificial Sequence
  synthesized oligonucleotide
accacactgcttcca

SEQ ID NO: 13 - DNA, 19 bp, Artificial Sequence
  synthesized oligonucleotide
agtggatgtctatgcttat